(19) United States
(12) Patent Application Publication
(10) Pub. No.: US 2017/0016867 A1
(43) Pub. Date: Jan. 19, 2017

(54) FLEXIBLE NITROGEN DIOXIDE GAS SENSOR BASED ON TUNGSTEN TRIOXIDE NANOPARTICLES COATED CARBON NANOTUBES-GRAPHENE OXIDE HYBRID AND METHOD FOR MANUFACTURING THE SAME

(71) Applicant: University of Ulsan Foundation For Industry Cooperation, Ulsan (KR)

(72) Inventor: Gwiy-Sang CHUNG, Ulsan (KR)

(21) Appl. No.: 14/927,911

(22) Filed: Oct. 30, 2015

(30) Foreign Application Priority Data
Jul. 13, 2015 (KR) 10-2015-0099331

(51) Int. Cl. G01N 33/00; H01B 1/04

(52) U.S. Cl. CPC G01N 33/0036; H01B 1/04; Y10S 977/752; Y10S 977/773; Y10S 977/892; Y10S 977/957; B82Y 40/00

(57) ABSTRACT

A flexible nitrogen dioxide sensor based on tungsten trioxide nanoparticles-loaded multi-walled carbon nanotubes-reduced graphene oxide (WO₃ NPs-loaded MWCNTs-RGO) hybrid on a polyimide/polyethylene terephthalate substrate. A viscous gel of the hybrid materials can be prepared by the assistance of α-terpineol. The fabricated sensor shows excellent sensing performance toward NO₂ which may have a maximum response of 17% (to 5 ppm), a limit of detection of 1 ppm, and relatively short response/recovery time (7/15 min). The sensor may exhibit excellent mechanical flexibility and sensing properties at room temperature without any significant performance degradation even at a curvature angle of 90° and after 10⁶ times of bending/relaxing processes. Low cost, light weight and mechanical robustness of the proposed WO₃ NPs-MWCNTs-RGO hybrid based sensor can be a promising element for the development of flexible NO₂ gas sensors having higher performance.

### CROSS-REFERENCES TO RELATED APPLICATIONS

This application claims the benefits of Korean Patent Application Number 1 0-2015-009933 filed on Jul. 13, 2015, at the Korean Intellectual Property Office, the disclosures of which are incorporated herein in their entirety by reference.

### BACKGROUND

1. Technical Field

The present disclosure relates to a flexible nitrogen dioxide (NO₂) gas sensor based on a tungsten trioxide nanoparticles (WO₃ NPs) coated carbon nanotubes (CNTs)-graphene oxide (GO) hybrid and a method for manufacturing the same, and more particularly to a flexible NO₂ gas sensor based on a WO₃ NPs coated CNTs-GO hybrid having higher performance for NO₂ gas detection, fabricated by using a WO₃ NPs-loaded multi-walled carbon nanotubes (MWCNTs)-reduced graphene oxide (RGO) hybrid where WO₃ NPs are added to a mixture solution of MWCNTs and RGO, and a method for manufacturing the same.

2. Description of the Related Art

Over the last few decades, carbon materials, such as one-dimensional (1D) carbon nanotubes (CNTs) and 2D/3D graphene, have been investigated extensively in the fields of electronics, field emission, energy, and sensors (A. K. Geim, Graphene: Status and Prospects, Science 324 (2009)1530-1534; Y. Ma et al., Three-dimensional graphene networks: synthesis, properties and applications, Natl. Sci. Rev. 2 (2014) doi: 10.1093/nsr/nwu072; W. Choi et al., Synthesis of Graphene and Its Applications: A Review, Crit. Rev. Solid State Mater. Sci. 35 (2010) 52-71; M. Paradise et al., Carbon nanotubes-Production and industrial applications, Mater. Des. 28 (2007) 1477-1489; T. Marek, Analytical applications of carbon nanotubes: a review, TrAC, Trends Anal. Chem. 25 (2006) 480-489). In recent years, MWCNTs and graphene nanosheets (GO and/or RGO) have attracted a great deal of attention as potential sensing elements to detect various gases (for example, NO₂, NH₃, H₂O, H₂, CO, etc.) due to their high surface area, high electrical conductivity, excellent carrier mobility, outstanding mechanical flexibility, and trace level detection ability at room temperature(W. Li et al., Reduced graphene oxide electrically contacted graphene sensor for highly sensitive nitric oxide detection, ACS nano 5 (2011) 6955-6961; T. Zhang et al., Recent progress in carbon nanotube-based gas sensors, Nanotechnology 19 (2008) 332001; W. Yuan et al., Graphene-based gas sensors, J. Mater. Chem. A 1 (2013) 10078-10091; S. G. Wang et al., Multi-walled carbon nanotube-based gas sensors for NH3 detection, Diamond Relat. Mater. 13 (2004) 1327-1332). Moreover, electron confinement due to their nano-dimensions provides unique directional pathways for flow of charge carriers, and thus enhances the sensing performance. In comparison to MWCNTs, RGO has higher active sights for adsorption of gas molecules. On the other hand, MWCNTs possess excellent mechanical flexibility along with high electrical properties (K. R. Douglas et al., Graphene versus carbon nanotubes for chemical sensor and fuel cell applications, Analyst 135 (2010) 2790-2797).

It was reported that hybrid nanostructures (consisting of RGO and MWCNTs) can potentially display synergistic effects and enhanced sensing properties that are superior to their individual counterparts (K. Yu et al., Carbon Nanotube with Chemically Bonded Graphene Leaves for Electronic and Optoelectronic Applications, J. Phys. Chem. Lett. 2 (2011) 1556-1562; K. Adarsh et al., Hybrid carbon nanostructured ensembles as chemiresistive hydrogen gas sensors, Carbon 49 (2011) 227-236). However, RGO/CNTs hybrid based chemical/gas sensors suffer from shortcomings, such as low adsorption energy, poor selectivity and long recovery time. Doping of heteroatom into hexagonal carbon structure of RGO is considered as a possible means to overcome these limitations. However, complex, hazardous, and expensive synthesis techniques limit its applicability. Instead, decoration of a suitable metal or metal oxide as catalyst is a simpler, economical, and safer way to deal with these problems.

A number of metal oxides (such as WO₃, SnO₂, ZnO, and In₂O₃)-loaded RGO or MWCNTs hybrid based gas sensors have already been reported in the literature as a promising development. Among these widely investigated metal oxides, recently, WO₃ has attracted great attention for its distinctive sensing properties toward numerous gases, such as NO₂, H₂S, and NH₃ (J. Qin et al., Graphene-wrapped WO3 nanoparticles with improved performances in electrical conductivity and gas sensing properties, J. Mater. Chem. 21 (2011) 17167-17174). WO₃ is a 2D n-type semiconductor with a wide band gap (varying from 2.6 to 3.2 eV), in which the weak Vander-walls force (due to wide band gap) allows a large space between material layers. This space can provide rooms for adsorption of gas molecules, and thus increase the sensor response value (L. Xu et al., Agx @ WO3 core-shell nanostructure for LSP enhanced chemical sensors, Sci. Rep. 4 (2014) 6745-6752). In general, detection principle of resistivity-type gas sensors is derived from the variation of conductance of sensing elements, while exposed to target gases. Therefore, the conductivity of WO₃ itself plays an important role in its sensing performance. It is believed that a combination of WO₃ with carbonaceous materials may have the possibility to reduce the activation energy of sensing layers and thus improve the adsorption capability.

Nitrogen dioxide (NO₂) is an oxidizing gas accompanied with an irritating odor. This typical air pollutant, which is usually produced by the combustion of fossil fuels, is very harmful to human beings and threatens the environment. In addition, it is also responsible for acid rain, global warming and the production of ozone (O₃). As a consequence, for environmental and personal safety purposes, it is important to develop a highly competent NO₂ gas sensor that can reliably detect and monitor this pollutant, even at a very low concentration. Besides, in recent years, research attention of conventional solid state gas sensor fabrication methods is diverted to cheap, light, and flexible device structures to facilitate wearable and portable gas sensor applications (M. C. Mcalpine et al., Highly ordered nanowire arrays on plastic substrates for ultrasensitive flexible chemical sensors, Nat. Mater. 6 (2007) 379-384; S. Claramunt et al., Flexible gas sensor array with an embedded heater based on metal decorated carbon nanofibres, Sens. Actuators B 187 (2013) 401-406). To meet the demand, flexible substrates (such as polyimide (PI), polyethylene terephthalate (PET), polydimethylsiloxane (PDMS), and polyethylene naphthalate (PEN)) have received enormous interests because of their low cost, mechanical stability, and biocompatibility. Among the various reported flexible substrates, polyimide (PI) has excellent thermal and chemical stability, high dielectric constant, and low coefficients of thermal expansion(W. A. MacDonald, Engineered films for display technologies, J. Mater. Chem. 14 (2004) 4-10; S. Walia et al., Flexible metasurfaces and metamaterials: A review of materials and fabrication processes at micro- and nano-scales, Appl. Phy. Rev. 2 (2015) 011303).

Recently, Cho et al. (B. Cho et al., Graphene-based gas sensor: Metal decoration effect and application to a flexible device, J. Mater. Chem. C 2 (2014) 5280-5285) reported an Al-coated multilayer graphene film on PI substrate based flexible sensor. The fabricated sensor showed enhanced NO₂ response at 150° C. with good flexibility (up to 10⁴ bending cycles) and good stability (to 3 months). Huang et al.(L. Huang et al., Fully printed, rapid-response sensors based on chemically modified graphene for detecting NO2 at room temperature, Appl. Mater. Inter. 6 (2014) 7426-7433) demonstrated a flexible NO₂ sensor based on sulphur doped RGO coated with Ag nanoparticles (Ag—S-RGO) on polyimide (PI) substrate with enhanced response and recovery time (0.16 s and 0.33 s). Su et al. (G. P. Su et al., Flexible NO₂ sensors fabricated by layer-by-layer covalent anchoring and in situ reduction of graphene oxide, Sens. Actuators B 190 (2014) 865-872) reported an in-situ reduction of graphene oxide (GO) based flexible NO₂ sensor on PET substrate with response and recovery time of 7 min and 28 min, respectively. Choi et al. (H. Choi et al., Flexible NO₂ gas sensor using multilayer graphene films by chemical vapor deposition, Carbon Lett. 14 (2013) 186-189) reported a flexible NO₂ sensor by CVD grown multilayer graphene film with a response time of 30 min. However, this group failed to achieve full recovery of the response. In addition to 3D graphene foam by CVD (C. Lee et al., Flexible-dimensional graphene foam-based NO₂ gas sensors, ECS trans. 61 (2014) 79-83), CNTs/RGO (H. Y. Jeong et al., Flexible room-temperature NO₂ gas sensors based on carbon nanotubes/reduced graphene hybrid films, Appl. Phys. Lett. 96 (2010) 213105-3) by self-assembly of multiwall carbon nanotubes (P. G. Su et al., Fabrication of flexible NO2 sensors by layer-by-layer self-assembly of multi-walled carbon nanotubes and their gas sensing properties, Sens. Actuators B 139 (2009) 488-493) and grapheme (C. Lee et al., Graphene-based flexible NO2 chemical sensors, Thin Solid Films 520 (2012) 5459-5462) have been studied for flexible NO₂ sensors. However, partial recovery, long recovery time, less stability in highly flexible environment, poor selectivity, and synthesis process complexity are still great challenges.

### SUMMARY

The present disclosure is directed to provide a flexible NO₂ gas sensor based on a WO₃ NPs coated CNTs-GO hybrid by using a WO₃ NPs-loaded MWCNTs-RGO hybrid synthesized via a ready solution process.

It was expected that according to a flexible NO₂ gas sensor based on a WO₃ NPs coated CNTs-GO hybrid of the present disclosure, the synthesized hybrid on PI substrate would enhance NO₂ sensing performance (in terms of response value, response/recovery time, and selectivity) including mechanical flexibility and improved performance stability at extreme mechanical deformation. A flexible NO₂ gas sensor based on a WO₃ NPs-loaded MWCNTs-RGO hybrid has not yet been reported in the literature. The fabricated gas sensor was evaluated systematically in terms of the sensor response at different material ratios, various bending angles, and several times of bending/relaxing processes.

According to an embodiment of the present disclosure, there is provided a flexible NO₂ gas sensor based on a WO₃ NPs coated CNTs-GO hybrid, which detects NO₂ gas and is manufactured by using a WO₃ NPs-loaded MWCNTs-RGO hybrid where MWCNTs, RGO, and WO₃ NPs are mixed at a proper mixing ratio.

According to an embodiment of the present disclosure, the MWCNTs and the RGO are preferably mixed with an assistance of α-terpineol to prepare a hybrid mixture solution before adding WO₃ NPs thereto.

According to an embodiment of the present disclosure, the MWCNTs, the RGO, and the WO₃ NPs in the hybrid are preferably mixed at a ratio of 3:1:2 by weight.

According to an embodiment of the present disclosure, the hybrid mixture solution is preferably dropped on a space between two gold (Au) electrodes attached on a polyimide (PI)/Si substrate, away from each other at a fixed distance and dried, to prepare the sensor.

According to an embodiment of the present disclosure, the gas sensor with the mixing ratio preferably shows a maximum response value of 17% at an annealing temperature of 200° C.

According to an embodiment of the present disclosure, the gas sensor preferably exhibits a limit of detection (LOD) of 1 ppm and a detection range of 1 to 25 ppm.

According to an embodiment of the present disclosure, the gas sensor preferably exhibits sensing performance after certain times of bending/relaxing cycles and at a fixed curvature angle.

According to an embodiment of the present disclosure, there is provided a method for manufacturing a flexible NO₂ gas sensor based on a WO₃ NPs coated CNTs-GO hybrid including the following steps: preparing a starting solution by mixing MWCNTs and synthesized GO powders with an assistance of α-terpineol; adding WO₃ NPs to the starting solution at a fixed mixing ratio with the MWCNTs and the GO; dropping the starting solution on a space between two gold (Au) electrodes, away from each other at a fixed distance, attached on a polyimide (PI)/Si substrate made of a PI tape and a Si substrate, and drying, and then performing annealing; and removing the PI tape from the PI/Si substrate and transferring the PI tape on to a polyethylene (PET) substrate.

According to an embodiment of the present disclosure, the MWCNTs, the GO, and the WO₃ NPs are preferably mixed at a ratio of 3:1:2 by weight.

According to an embodiment of the present disclosure, the PI/Si substrate on which the gold (Au) electrodes are attached and the starting solution is dried is preferably annealed at 200 ° C.

According to a flexible NO₂ gas sensor based on a WO₃ NPs coated CNTs-GO hybrid and a method for manufacturing the same of the present disclosure, the flexible NO₂ gas sensor has outstanding mechanical flexibility, durability, and robustness, and also shows outstanding NO₂ gas sensing performance by having a shorter recovery time and higher response value.

Also, according to a flexible NO₂ gas sensor based on a WO₃ NPs coated CNTs-GO hybrid and a method for manufacturing the same of the present disclosure, the flexible NO₂ gas sensor shows a stable response value magnitude where no deformation occurs even after numerous bending/relaxing processes.

Also, according to a flexible NO₂ gas sensor based on a WO₃ NPs coated CNTs-GO hybrid and a method for manufacturing the same of the present disclosure, since a sensor response value increases as relative humidity increases, the flexible NO₂ gas sensor shows outstanding sensing performance even in a moist environment.

### BRIEF DESCRIPTION OF THE DRAWINGS

FIG. 1(a) is a schematic diagram illustrating a fabricated gas sensor according to an embodiment of the present disclosure.

FIG. 1(b) is an optical image of a fabricated gas sensor according to an embodiment of the present disclosure.

FIG. 2(a) shows an SEM image of a WO₃ NPs-loaded MWCNTs-RGO hybrid where WO₃ NPs, MWCNTs, and RGO are mixed at a mixing ratio of 3:0.5:1 (S1).

FIG. 2(b) shows an SEM image of a WO₃ NPs-loaded MWCNTs-RGO hybrid where WO₃ NPs, MWCNTs, and RGO are mixed at a mixing ratio of 3:1:2 (S2).

FIG. 2(c) shows an SEM image of a WO₃ NPs-loaded MWCNTs-RGO hybrid where WO₃ NPs, MWCNTs, and RGO are mixed at a mixing ratio of 3:2:3 (S3).

FIG. 2(d) shows an SEM image of a WO₃ NPs-loaded MWCNTs-RGO hybrid S2 after several times of bending/relaxing.

FIG. 3(a) shows a TEM image of S2 hybrid at low magnification.

FIG. 3 (b) shows a TEM image of S2 hybrid at high magnification.

FIG. 3(c) shows an HRTEM image at an interface between WO₃ NP and MWCNT.

FIG. 3(d) shows an EDS spectrum of S2 hybrid.

FIG. 4 shows XRD patterns of a WO₃ NPs-loaded MWCNTs-RGO hybrid at different annealing temperatures.

FIG. 5(a) shows Raman spectra of pure MWCNTs, RGO, and a MWCNTs-RGO hybrid.

FIG. 5(b) shows Raman spectra of S1, S2, and S3 hybrids.

FIG. 6 shows BET results of pure WO₃, a WO₃-MWCNTs hybrid, and a WO₃-MWCNTs-RGO hybrid.

FIG. 7 shows response values of S1, S2, and S3 sensors at different annealing temperatures toward 5 ppm NO₂ gas.

FIG. 8 shows real time resistance changes of S1, S2, and S3 sensors toward NO₂.

FIG. 9 shows comparison of response values among pure MWCNTs, a WO₃-MWCNTs hybrid, and a WO₃ NPs-loaded MWCNTs-RGO hybrid.

FIG. 10 shows transient response values of S2 hybrid toward 5 ppm NO₂ gas at different bending angles.

FIG. 11 shows response/recovery time characteristics of S2 hybrid toward 5 ppm NO₂ gas.

FIG. 12(a) shows sensor response value vs. NO₂concentration curve at different bending angles.

FIG. 12(b) shows sensor response value vs. NO₂concentration curve after several times of bending/relaxing.

FIG. 13 shows humidity effect on a sensor for NO₂concentration of 5 ppm.

FIG. 14 is a selectivity graph of a fabricated sensor toward 5 ppm NO₂ and 1000 ppm test gases.

### DETAILED DESCRIPTION

Hereinafter, preferred example embodiments of a flexible NO₂ gas sensor based on a WO₃ NPs coated CNTs-GO hybrid and a method for manufacturing the same according to the present disclosure are described in detail with reference to the accompanying drawings. However, it should be noted that the present disclosure is not limited to the embodiments described below but may come with a diversity of embodiments. The embodiments are provided only to complete the present disclosure and help those skilled in the art fully understand the scope of the present disclosure.

A flexible NO₂ gas sensor based on a WO₃ NPs coated CNTs-GO hybrid and a method for manufacturing the same according to the present disclosure are described in detail. The fabrication of a flexible NO₂ gas sensor according to the present disclosure is described through the following experiment.

### 1. Experiment

### 1.1 Materials Synthesis and Sensor Fabrication

All of the chemicals used in the synthesis process were of analytical grade purchased from Sigma Aldrich, Dongwoo Fine-Chem., and Dae Jung Chem. & Inds. Co. Ltd., and were used without further purification.

Synthesis of WO₃NPs and graphene oxide (GO): Tungstatedihydrate (Na₂WO₄.2H₂O) and cetyltrimethylammonium bromide (CTAB) were used as precursor and surfactant, respectively. In a typical process, 10 mL of CTAB (0.5 M) and 10 mL of Na₂WO₄.2H₂O (1.5 M) aqueous solution were added to 20 mL of de-ionized (DI) water using vigorous stirring. 1 mL of HCl (3 M) was subsequently added drop-wise to the solution to obtain a pH level around 3. The as-prepared solution was then transferred into a sealed Teflon autoclave and heated at 120° C. for 12 h. The final product was collected after several times of washing with ethanol-DI water (1:1) and dried overnight at 60° C. GO solution was synthesized by modified Hummer's method as describes in elsewhere(W. S. Hummers Jr. et al., Preparation of graphitic oxide, J. Am. Chem. Soc. 80 (1958), 1339-1339; D. T. Phan et al., Photodiodes based on graphene oxide-silicon junctions, Solar Energy 86 (2012) 2961-2966). GO solution was dried at 45° C. for 48 h to obtain fine GO powders.

Synthesis of a WO₃ NPs-loaded MWCNTs-RGO hybrid: Commercial MWCNTs (Φ=4.5 to 5 nm; 1=3 to 6 μm) and synthesized GO powders were mixed with an assistance of α-terpineol using sonication treatment for 1 h, to prepare a starting solution. Thereafter, 3 mg of WO₃ NPs powders were added to the starting solution and subjected to sonication treatment for another 1 h. To obtain an optimum material ratio, three different hybrid samples were prepared by varying the amounts of MWCNTs and GO to WO₃ (WO₃:MWCNTs:GO=(a) 3:0.5:1; (b) 3:1:2; (c) 3:2:3).

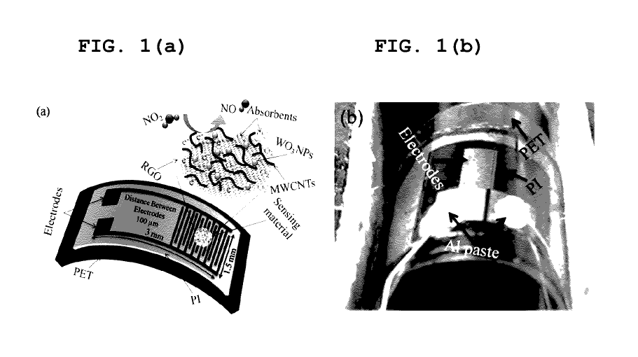

Sensor fabrication: To fabricate a sensor, a commercial PI tape was attached on a Si substrate. Two finger electrodes of gold (distance: 100 μm) were deposited on the top of a PI/Si substrate using photolithography and radio frequency magnetron sputtering. The as-prepared hybrid was then drop casted between the finger electrodes and placed on a hot plate at 100° C. for drying. Afterward, each sample was annealed at different temperatures (100, 150, 200, and 250° C.) for 1 h. For better clarity, sensors were labeled as S1 (WO₃ NPs-MWCNTs-RGO =3:0.5:1); S2 (WO₃ NPs-MWCNTs-RGO=3:1:2) and S3 (WO₃ NPs -MWCNTs-RGO=3:2:3). Finally, the PI tape was carefully peeled-out from the Si substrate and transferred on to a PET substrate. The schematic diagram and the optical image of the fabricated sensor are shown in FIG. 1(a) and FIG. 1(b), respectively.

### 1.2 Characterization

Phase transition analysis was carried out by an X-ray diffractometer (XRD) (Ultima IV, Rigaku Corporation) with Cu Kα (λ=0.154056 nm) radiation and a 2θ scanning range of 10 to 70°. The surface morphology and elemental characterizations of the as-prepared hybrids were examined by field emission scanning electron microscopy (FESEM, JEOL-JSM-7600F), transmission electron microscopy (TEM, JEOL JEM-2100F), high-resolution TEM (HRTEM) and energy dispersive spectroscopy (EDS, JEOL JEM-2100F). Raman spectra were acquired through a WITec spectrometer with 532 nm laser excitation in order to detect possible structural properties and quality of the synthesized hybrid materials. The Brunauer Emmett and Teller (BET) analysis of pure MWCNTs, WO₃-MWCNTs, and WO₃-MWCNTs-RGO was measured by nitrogen adsorption at 77 K temperature to observe specific surface area (SSA(_{BET})) of each material.

The gas sensing characterizations were carried out at room temperature in an open air environment. A computerized mass flow controller system (GMC 1200 ATOVAC Co., Ltd.) was used to vary the NO₂ gas concentration. A gas mixture (synthetic air and NO₂) was delivered on the top of a sensor device at a constant flow rate of 50 standard cubic centimeters per minute (sccm) with different NO₂ concentrations. Gas concentration was controlled and measured by the following equation 1:

$${Gas}_{com} ( ppm ) = \frac{{Flowrate}_{air} + {Flowrate}_{gas}}{Totalflowrate}$$

A sensor response value was calculated by the following equation 2:

$$S ( \% ) = \frac{{R}_{a} - {R}_{g}}{{R}_{a}} \times 100 ,$$

where S (%) denotes a sensor response value in percentage, Rₐ is the resistance of the sensor in air, and R_{g} is the resistance after exposure to a certain amount of NO₂. The response time and recovery time of the sensor were defined as the time taken to reach 90% of the total resistance change.

### 2. Results and Discussions

### 2.1 Crystal Structure and Morphology

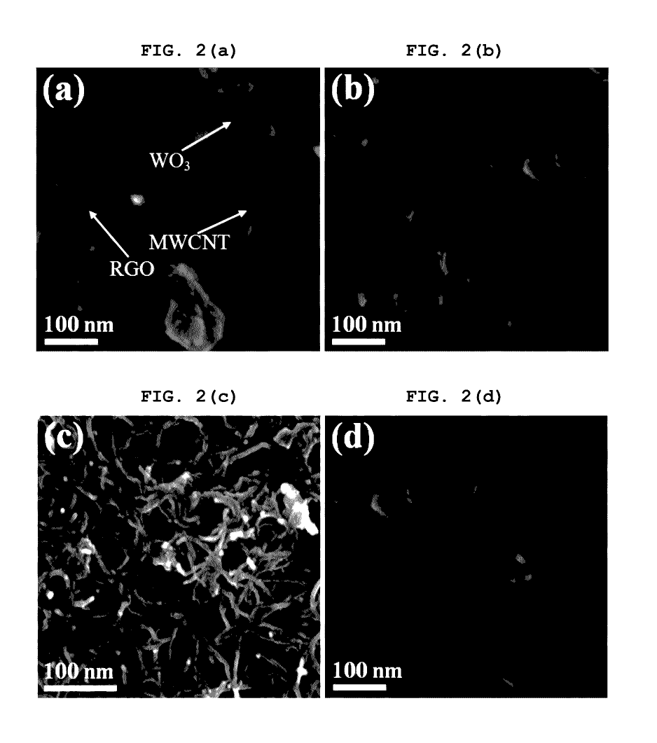

FIG. 2 shows FESEM images of WO₃ NPs-loaded MWCNTs-RGO hybrids (samples S1, S2 and S3 annealed at 200° C.). All of the observed samples include irregularly shaped WO₃ nanoparticles, long isolated MWCNTs, and thin RGO nanosheets. A small amount of aggregation was frequently observed in the samples that can probably be accounted for the presence of different materials with high density. WO₃ nanoparticles were more visible in sample S1 (FIG. 2(a)) compared to other samples (S2 (FIGS. 2(b)) and S3 (FIG. 2(c))). However, a large amount of aggregation occurred in sample S3 due to the presence of large numbers of RGO and MWCNTs that unsurprisingly covered the WO₃ nanoparticles. The samples were annealed at different temperatures (100 to 250° C.) and investigated again. The variation in the annealing temperature did not reveal any changes in the morphology of the hybrid samples. Later on, samples S1, S2 and S3 (annealed at 200° C.) were bended-relaxed repeatedly up to 10⁷ cycles and analyzed by FESEM. FIG. 2(d) represents the FESEM image of sample S2 after 10⁷ times of bending and relaxing. No significant deformation or degradation was observed after several times of bending and relaxing. This phenomenon might be attributed to exceptional mechanical robustness and outstanding flexibility properties of the MWCNTs and RGO in the hybrids and superior bending between the hybrids and PI substrate.

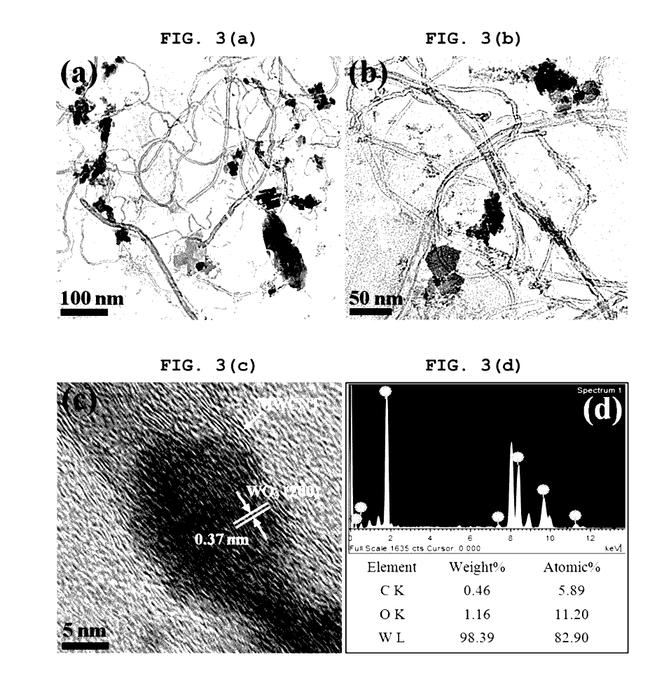

For detailed morphological investigations, TEM analysis was carried out at different magnifications and shown in FIG. 3. FIG. 3(a) affirms the decoration of tiny sized WO₃ NPs on transparent RGO sheets and long MWCNTs. The average size of WO₃ NPs was estimated to 20 nm from TEM observations. However, WO₃ NPs with irregular shape was also observed, which can be attributed to the aggregation among the small sized particles. FIG. 3(b) shows the morphology of the as-prepared hybrid at high magnification. FIG. 3(c) presents the HRTEM image which indicates rightly fitted WO₃ NPs on MWCNTs with continuity of lattice fringes of WO₃ NPs and MWCNTs. The measured spacing between adjacent lattice fringes is 0.37 nm, corresponding to (200) plane of WO₃. FIG. 3(d) represents the elementary analysis of the hybrid sample (S2) and confirms the presence of tungsten (W), carbon (C), and oxygen (O). This result presents the formation of high purity hybrid sample.

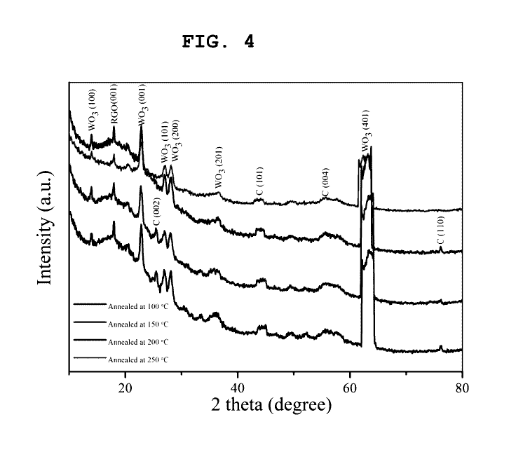

XRD was carried out to analyze the crystalline structure of S2 hybrid formed as crystals, at different annealing temperatures. FIG. 4 shows the well-structured crystalline nature of the synthesized hybrid. The different diffraction peaks appearing at 2θ=14.11°, 22.89°, 26.87°, 28.30°, 36.67°, and 62.33° corresponds to the hexagonal WO₃ (100), (001), (101), (200), (201), and (401) planes, respectively (h-WO₃, ICDD: 01-075-2187). Additionally, characteristic diffraction peak at 2θ=18.1° corresponds to RGO (002) plane, suggesting the partial reduction of GO. Furthermore, diffraction peaks at 2θ=43.66°, 55.35°, and 76.77° appeared on carbon (101), (004), and (110) planes, respectively (C, ICDD: 00-023-0064). No significant changes or shifts in peak position were observed in the spectrum after different annealing temperatures. However, some impurities peaks were removed at higher temperature leading to the purity of hybrid materials. The intensity of the peak of RGO at 250° C. was reduced as compared to 200° C. indicating low RGO contaminants. This ultimately causes the reduction in SSA of RGO.

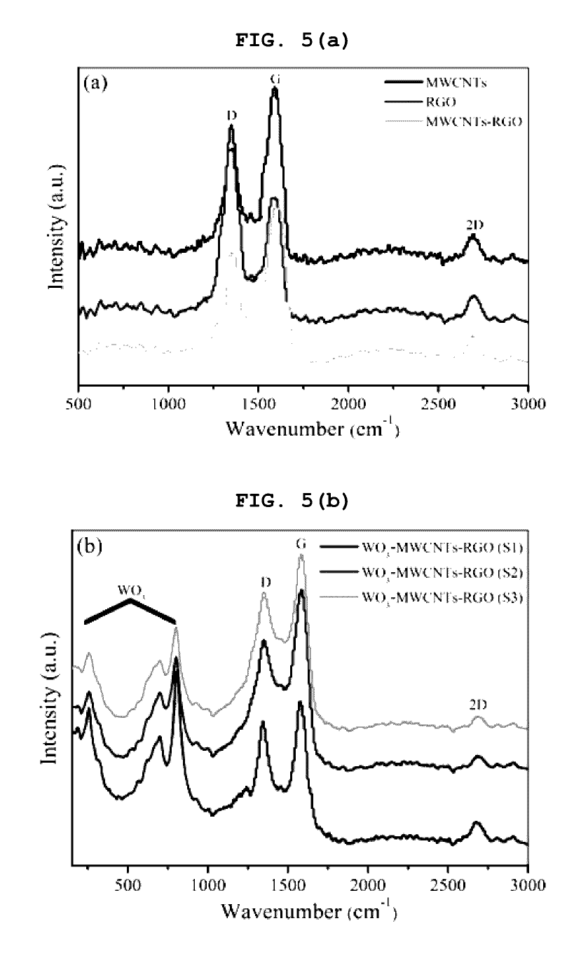

Raman spectroscopy was carried out to study the order/disorder of hexagonal carbon structure and the effect of WO₃ thereon. FIG. 5(a) represents Raman spectra of pure MWCNTs, RGO, and a MWCNTs-RGO hybrid. The D peaks of pure MWCNTs, RGO, and MWCNTs-RGO hybrid were observed between 1350 and 1355 cm⁻¹. Additionally, the G and 2D peaks of MWCNTs, RGO, and MWCNTs-RGO hybrid were observed between 1590 and 1598 cm⁻¹ and between 2689 and 2697 cm⁻¹, respectively. The low I_{d}/I_{g} ratio (0.90) of MWCNTs may be attributed to low disorder in carbon network. On the other hand, in RGO, high I_{d}/I_{g} ratio (1.15) and wide intensity peaks were observed, which may be ascribed as higher level of disorder between RGO flakes. The MWCNTs-RGO hybrid exhibits the lowest I_{d}/I_{g} ratio (0.87) with wide intensity peaks. The wide intensity peaks may be explained as disorder between MWCNTs and RGO hexagonal structure. Additionally, Raman spectra of WO₃-MWCNTs-RGO hybrids (S1, S2, and S3) were also investigated and shown in FIG. 5(b). The lower frequency band located at 260 cm⁻¹ may be attributed to the W-O-W bending vibrations, whereas the peaks observed at 701 and 803 cm⁻¹ may be assigned to the stretching modes of the W-O-W bonds(A. Esfandiar et al., Pd-WO3/reduced graphene oxid hierarchical nanostructures as efficient hydrogen sensors, Int. J. Hydrogen Energy 39 (2014) 8169-8179). A little increase in I_{d}/I_{g} ratio (0.88) was observed in samples S2 and S3, compared to a MWCNTs-RGO hybrid which might be caused by WO₃. Sample S1 exhibits a higher I_{d}/I_{g} ratio that may be caused due to the presence of high amount of WO₃.

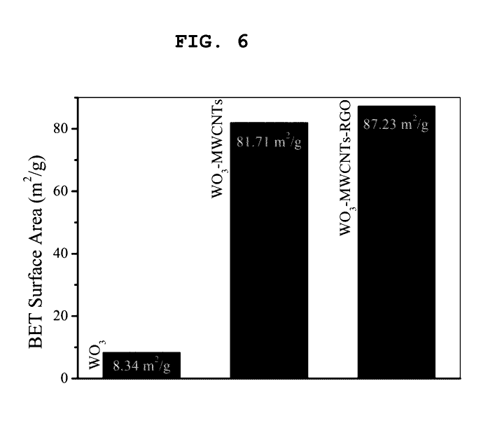

FIG. 6 shows the BET analysis of pure WO₃, WO₃-MWCNTs, and WO₃-MWCNTs-RGO (S2, annealed at 200° C.). It clearly reveals that WO₃-MWCNTs-RGO has a larger specific surface area (SSA) of 87.23 m²/g, whereas pure WO₃ and WO₃-MWCNTs have specific surface areas of 8.34 m²/g and 81.71 m²/g, respectively. When MWCNTs were added to pure WO₃, the addition increases the SSA (approximately 10 times), which might be attributed to high porosity of MWCNTs. Furthermore, when RGO was added to WO₃-MWCNTs, the addition further increases the SSA, which might be caused by the presence of the basal plane of RGO.

### 2.2 Gas Sensing Properties

The gas sensing properties of fabricated sensors were carried out in an open air environment at room temperature (20° C.). NO₂ is an oxidizing gas, which captures electrons and subsequently increases or decreases conductance of the sensing layer. In this particular case, when NO₂ gas interacts with the sensing layer, the interaction increases the conductance suggesting p-type behavior of the sensing layer (G. Lu et al., Reduced graphene oxide for room-temperature gas sensors, Nanotechnology 20 (2009) 445502-445511). The probable sensing mechanism of the fabricated sensor is explained in FIG. 1(a). When the sensor was placed in an open air environment, oxygen molecules enter and capture electrons from the surface of the sensing layer and leave oxygen absorbents (O₂⁻). When NO₂ molecules enter and interact with the sensing layer surface, they take electrons, are dissociated in the foam of NO, and leave oxygen absorbents (O₂⁻). The O₂⁻anion then becomes an active site to adsorb NO₂molecules. Subsequently, NO may not convert to NO₂ again after reacting with half of the O₂ molecules. This phenomenon continuously happened after NO₂ exposure.

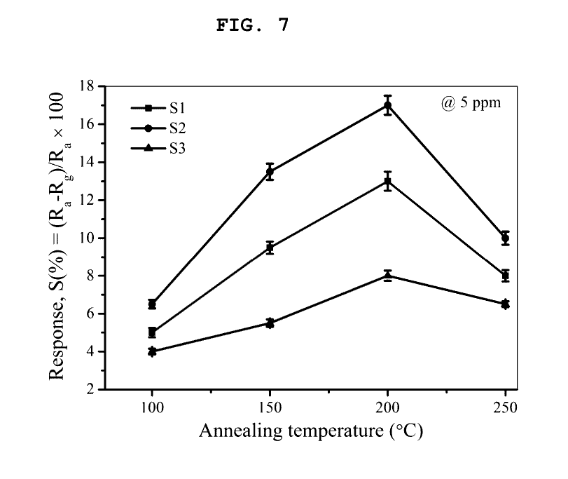

FIG. 7 shows the response value variations of samples S1, S2, and S3 checked in terms of different annealing temperatures toward 5 ppm NO₂ concentration. Sample S2 showed a maximum response value of 17% at an optimum annealing temperature of 200° C. This may be attributed to the high specific surface area of the synthesized material and formation of the depletion layer through p-n junctions between p-type MWCNTs/RGO and n-type WO₃ NPs. This depletion layer with excess of charges may play an important role to increase the sensor response value (S. Srivastava et al., Faster response of NO2 sensing in graphene-WO3 nanocomposites, Nanotechnology 23 (2012) 205501-205507; J. S. Lee et al., WO₃ nanonodule-decorated hybrid carbon nanofibers for NO2 gas sensor application, J. Mater. Chem. A 1 (2013) 9099-9106). After the exposure of NO₂, a number of charges were transferred from the specific region for NO₂molecules resulting in a dramatic increase in the sensor response. Annealing temperature also plays a significant role to enhance the sensor performance. At lower temperatures (100 and 150° C.), probably, GO was not completely reduced. In contrast, at a higher temperature (250° C.), RGO might be decomposed slightly (XRD shows a minimum decrease at intensity peak) and might cause reduction in SSA. It was supposed that at an optimum annealing temperature of 200° C., α-terpineol was fully removed and RGO exhibits maximum SSA.

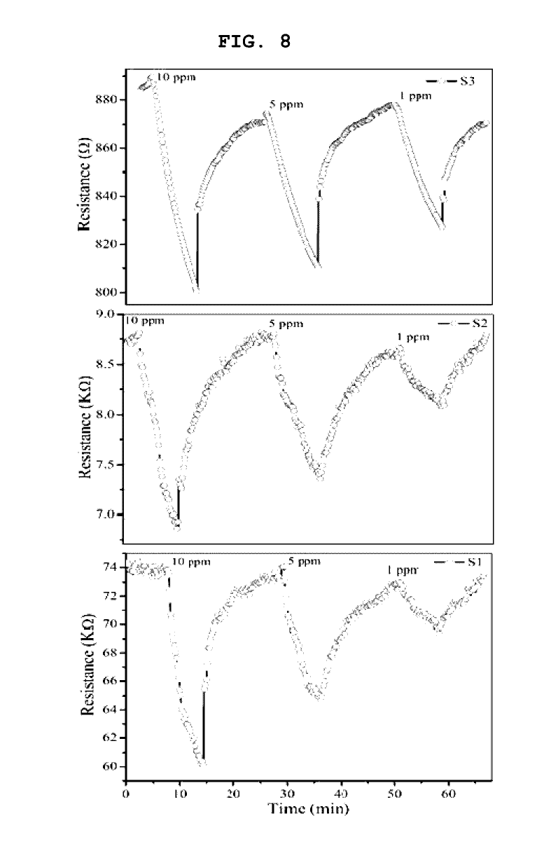

FIG. 8 shows the real time resistance changes of all the sensors (S1, S2, and S3, annealed at 200° C.) in terms of NO₂ gas concentration. It was observed that samples S1 and S3 have the highest and the lowest resistance values, respectively. This phenomenon might be caused by the variation in amounts of MWCNTs and RGO. The presence of higher amounts of MWCNTs and RGO in S3 resulted in higher conductivity, opposed to forming balanced p-n junctions, and lower selectivity to NO₂ gas. On the contrary, sample S1 contained a higher amount of WO₃ compared to the other samples that may create (O_{ads}⁻ or O²⁻_{ads} adsorbents on the sensing surface, and prevented NO₂ molecules from reacting at lower temperatures. In comparison to samples S1 and S3, S2 was endowed with suitable p-n junctions that created adequate active sites to adsorb a maximum number of gas molecules. In addition, larger SSA played an important role in response enhancement.

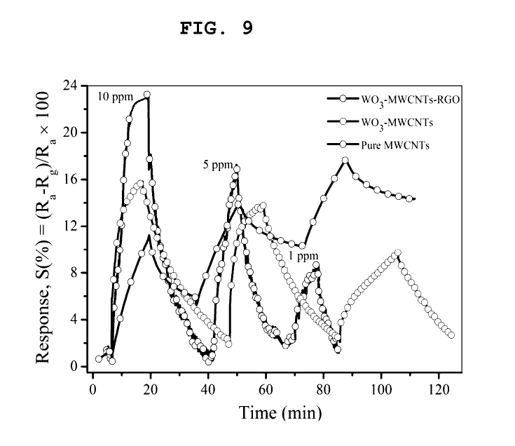

FIG. 9 shows the response properties of pure MWCNTs and WO₃-MWCNTs hybrid, compared to WO₃-loaded MWCNTs-RGO hybrid sensor (sample S2), in order to support the BET results. It was clearly observed that S2 sensor exhibits a highest response value along with a shorter recovery time, compared to pure MWCNTs and WO₃-MWCNTs hybrid sensor. The high SSA and enhanced charge transfer pathway were provided by the WO₃ NPs, RGO sheets, and MWCNTs network which facilitated the adsorption-desorption kinetics during sensor characterizations, hence showed better sensor performance. Moreover, a low response value and partial recovery of the pure MWCNTs sample might be attributed to the absence of p-n junction and high bending energy between carbon and NO₂ molecules(J. Li et al., Carbon nanotube sensors for gas and organic vapor detection, Nano Lett. 3 (2003) 929-933).

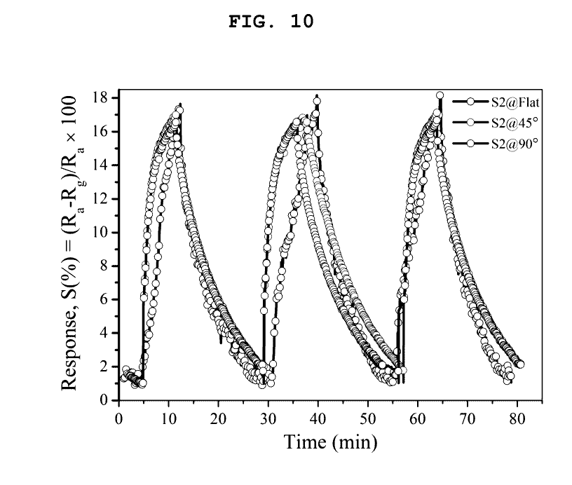

To investigate the reliability and mechanical flexibility, the fabricated sensor sample S2 was evaluated at different curvature angles (0 to 90° at room temperature. FIG. 10 shows the dynamic response values of S2 hybrid at situations un-bent (flat or 0°) and bent to 5 ppm NO₂. A response value degradation was observed at 45° (to 1.1%) and at 90° (to 1.7%) deformation, which might be attributed to the low bending energy and slight change between strained carbon atoms and NO₂molecules at deformed situation. This negligible drop in response value magnitude confirms the high mechanical robustness of the fabricated sensor. In addition, no significant change in cycle-to-cycle response value (drift to 0.3%) further confirms the reliability of the sensor.

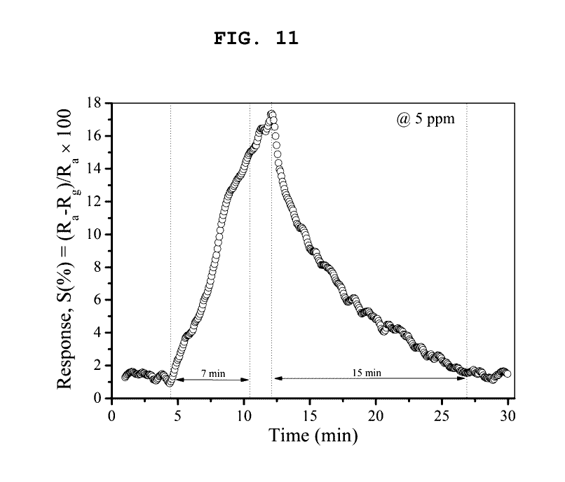

Furthermore, the fabricated sensor showed improved response-recovery time compared to reported results. FIG. 11 reveals that S2 sensor reached its maximum response value position within 7 minutes and returned to its initial position within 15 minutes. This short response-recovery time characteristics of the fabricated sensor might be accounted for by the addition of MWCNTs and RGO with WO₃NPs, in which low bending energy on the sensing surface (adsorption process) and quick elimination of NO₂ molecules (desorption process) are vital. However, mechanical deformation (bending angles from 0° to 90°) on the sensor during sensor characterization did not show any change in the response-recovery time behaviors.

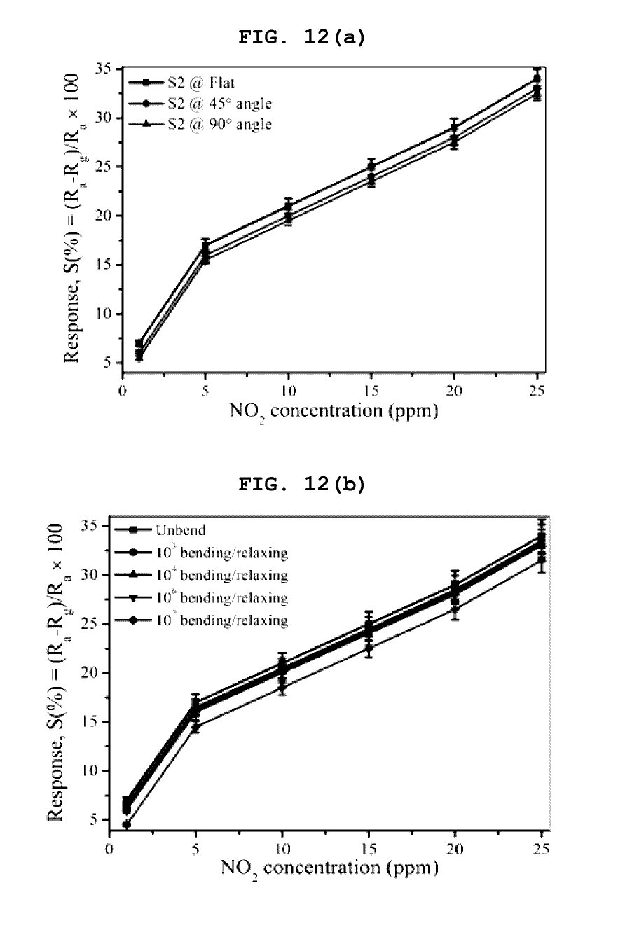

To examine the mechanical stability of the fabricated sensor, fatigue tests were carried out at different bending angles and after several times of bending-relaxing. FIG. 12(a) represents the response value variation of S2 sensor in terms of NO₂ concentration at different bending angles. The sensor exhibited a limit of detection (LOD) of 1 ppm and a detection range of 1 to 25 ppm along with good linearity behavior within the entire NO₂ concentration range. FIG. 12(b) shows the response value variation of S2 hybrid within 1 to 25 ppm NO₂ concentration after several times of bending-relaxing processes. No remarkable degradation in response value magnitude was observed up to 10⁶ times of bending and relaxing processes. This can be attributed to excellent mechanical flexibility of MWCNTs and RGO, the PI substrate which was not crushed or destroyed even after 10⁶ times of bending and relaxing, and excellent attachment of WO₃-MWCNTs-RGO network with tiny sized WO₃NPs. However, after 10⁷ repeated cycles, the response value magnitude was dropped to about 3%, which might be caused by lower attachment between the sensing layer and the substrate due to excessive mechanical stress on the sensor.

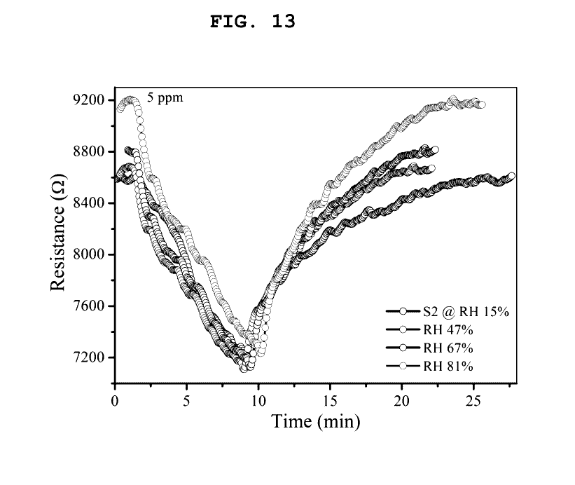

Furthermore, humidity effect on the sensor was investigated. Humidity is one of the influencing factors on gas sensing properties (E. Traversa, Ceramic sensors for humidity detection: the state-of-the-art and future developments, Sens. Actuators B 23 (1995) 135-156; C. Wang et al., Metal oxide gas sensors: sensitivity and influencing factors, Sensors 10 (2010)2088-2106). It was reported specifically for NO₂ sensing; both water and NO₂ molecules behave as an oxidizing agent, and consequently, result in an increase in sensor response value (W. Yuan et al., Graphene-based gas sensors, J. Mater. Chem. A 1 (2013) 10078-10091). FIG. 13 shows the change in sensor response value of the S2 sensor (toward 5 ppm NO₂) after introducing relative humidity (RH). The baseline resistance of the sensor was shifted to higher level with increasing humidity concentration. It was observed that the sensor response value was increased approximately 4% with a slight enhancement in recovery time at 81% RH.

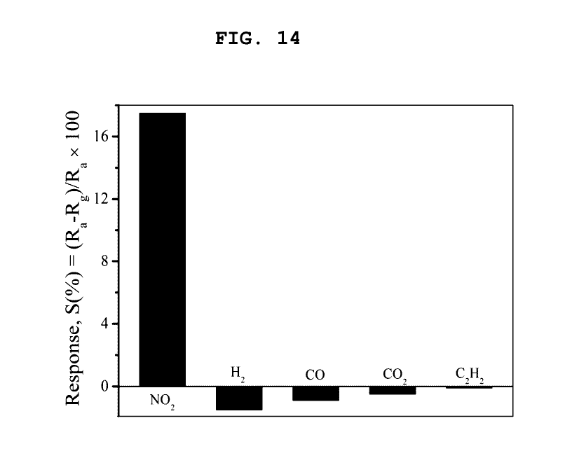

Finally, the selectivity of the fabricated sensor (S2) was investigated by exposing the sensor to different test gases at room temperature. FIG. 14 shows the selectivity histogram of the fabricated sensor toward 5 ppm NO₂ and 1000 ppm test gases, including carbon monoxide, carbon dioxide, and acetylene. As expected, S2 sensor showed better selectivity properties toward NO₂, which might be attributed to superior adsorption capability of WO₃ and carbon materials toward NO₂ molecules.

### 3. Conclusions

In summary, fabrication and characterization of a high-performance NO sensor with enhanced sensing properties and excellent mechanical flexibility have been investigated at room temperature. The WO₃ NPs-loaded MWCNTs-RGO hybrid at a ratio of 3:1:2 (sample S2) showed a maximum response value of 17% (5 ppm) to NO₂ (a relatively short response-recovery time (7/15 minutes), an LOD of 1 ppm, and a detection range of 1 to 25 ppm. The sensor also showed exceptional mechanical flexibility and excellent repeatability at different bending angles with a negligible drift of 1.7% (at 90°) and approximately 3% degradation after 10⁷ times of bending-relaxing processes. Less humidity effect (up to 67% RH) on the sensing surface and the excellent selectivity demonstrate that the sensor fabricated according to the present disclosure may be a suitable candidate for the fabrication of high-performance and practical NO₂ sensor and be used in various sensors (vehicles, aircraft, aeronautics, and portable electronics).

As described above, preferred example embodiments of a flexible NO₂ gas sensor based on a WO₃ NPs coated CNTs-GO hybrid and a method for manufacturing the same according to the present disclosure are described with reference to the accompanying drawings. However, it should be noted that the present disclosure is not limited to the described embodiments and drawings but may come with a diversity of embodiments by those skilled in the art within the inventive concept of the present disclosure.

What is claimed is:

1. A flexible nitrogen dioxide (NO₂) gas sensor based on a tungsten trioxide nanoparticles (WO₃ NPs) coated carbon nanotubes (CNTs)-graphene oxide (GO) hybrid, the flexible gas sensor detecting NO₂ gas being manufactured by using a tungsten trioxide nanoparticles (WO₃ NPs)-loaded multi-walled carbon nanotubes (MWCNTs)-reduced graphene oxide (RGO) hybrid where multi-walled carbon nanotubes (MWCNTs), reduced graphene oxide (RGO), and tungsten trioxide nanoparticles (WO₃ NPs) are mixed at a proper mixing ratio.

2. The flexible nitrogen dioxide (NO₂) gas sensor of claim 1, wherein the multi-walled carbon nanotubes (MWCNTs) and the reduced graphene oxide (RGO) are mixed with an assistance of α-terpineol to prepare a hybrid mixture solution, before adding the tungsten trioxide nanoparticles (WO₃ NPs) thereto.

3. The flexible nitrogen dioxide (NO₂) gas sensor of claim 1, wherein the multi-walled carbon nanotubes (MWCNTs), the reduced graphene oxide (RGO), and the tungsten trioxide nanoparticles (WO₃ NPs) in the hybrid are mixed at a ratio of 3:1:2 by weight.

4. The flexible nitrogen dioxide (NO₂) gas sensor of claim 2, wherein the hybrid mixture solution is dropped on a space between two gold (Au) electrodes, away from each other at a predetermined distance, deposited on a polyimide (PI)/Si substrate and dried to prepare the sensor.

5. The flexible nitrogen dioxide (NO₂) gas sensor of claim 4, wherein the gas sensor with the mixing ratio shows a maximum response value of 17% at an annealing temperature of 200° C.

6. The flexible nitrogen dioxide (NO₂) gas sensor of claim 4, wherein the gas sensor exhibits a limit of detection (LOD) of 1 ppm and a detection range of 1 to 25 ppm.

7. The flexible nitrogen dioxide (NO₂) gas sensor of claim 4, wherein the gas sensor exhibits sensing performance after certain times of bending/relaxing cycles and at a certain curvature angle.

8. A method for manufacturing a flexible nitrogen dioxide (NO₂) gas sensor based on a tungsten trioxide nanoparticles (WO₃ NPs) coated carbon nanotubes (CNTs)-graphene oxide (GO) hybrid, the method comprising:
preparing a starting solution by mixing multi-walled carbon nanotubes (MWCNTs) and synthesized graphene oxide (GO) powders with an assistance of α-terpineol;
adding tungsten trioxide nanoparticles (WO₃ NPs) to the starting solution at a predetermined mixing ratio with the multi-walled carbon nanotubes (MWCNTs) and the graphene oxide (GO);
dropping the starting solution on a place between two gold (Au) electrodes, away from each other at a predetermined distance, deposited on a polyimide (PI)/Si substrate made of a PI tape and a Si substrate, and drying, and then performing annealing; and
removing the polyimide (PI) tape from the PI/Si substrate and transferring the PI tape on to a polyethylene (PET) substrate.

9. The method of claim 8, wherein the multi-walled carbon nanotubes (MWCNTs), the graphene oxide (GO), and the tungsten trioxide nanoparticles (WO₃ NPs) are mixed at a ratio of 3:1:2 by weight.

10. The method of claim 8, wherein the polyimide (PI)/Si substrate on which the gold (Au) electrodes are deposited and the starting solution is dried is annealed at 200° C.
